(19) United States
(12) Patent Application Publication
(10) Pub. No.: US 2014/0275582 A1
(43) Pub. Date: Sep. 18, 2014

(54) CHIRAL 2-ARYLPROPYL-2-SULFINAMIDE AND CHIRAL N-2-ARYLPROPYL-2-SULFINYLIMINES AND SYNTHESIS THEREOF

(71) Applicant: Guigen LI, Lubbock, TX (US)

(72) Inventor: Guigen LI, Lubbock, TX (US)

(73) Assignees: Texas Tech University, Lubbock, TX (US); Nanjing University, Institute of Chemistry and Biomedical Sciences, Nanjing (CN)

(21) Appl. No.: 14/207,390

(22) Filed: Mar. 12, 2014

Related U.S. Application Data
(60) Provisional application No. 61/784,300, filed on Mar. 14, 2013.

(51) Int. Cl. C07D 307/52; C07C 313/02; C07C 313/06

(52) U.S. Cl. CPC C07D 307/52; C07C 313/06; C07C 313/02
USPC 549/491; 564/102; 564/275; 564/270; 564/271

(57) ABSTRACT

Provided herein are novel chiral sulfinamide and imine compounds. Also provided herein are methods of synthesizing novel chiral sulfinamide and imine compounds comprising simplified purification methods when compared to prior methods. The novel chiral sulfinamide and imine compounds are useful, for example, in the synthesis of complex natural products and pharmaceutical important compounds.

### CROSS-REFERENCE TO RELATED APPLICATION

This application claims the benefit of U.S. provisional application No. 61/784,300, filed on Mar. 14, 2013, which is incorporated by reference herein in its entirety.

### STATEMENT REGARDING FEDERALLY SPONSORED RESEARCH OR DEVELOPMENT

This invention was made with government support under R21DA031860-01 awarded by the National Institutes of Health. The government has certain rights in the invention.

### FIELD

Provided herein are novel chiral sulfinamide and imine compounds. Also provided herein are methods of synthesizing novel chiral sulfinamide and imine compounds comprising simplified purification methods when compared to prior methods. The novel chiral sulfinamide and imine compounds are useful, for example, in the synthesis of complex natural products and pharmaceutically important compounds.

### BACKGROUND

Imine chemistry plays a pivotal role in producing biologically active amine compounds. F. A. Davis, P. Zhou, B. C. Chen, Chem. Soc. Rev. 1998, 27, 13-18). The asymmetric nucleophilic addition reaction on imines produces a wide variety of chiral amine containing moieties. F. A. Davis, B. Yang, J. Am. Chem. Soc. 2006, 127, 8938-8407; F. A. Davis, R. E. Reddy, J. M. Szewczyk, G. V. Reddy, P. S. Potonovo, H. Zhang, D. T. Reddy, P. Zhou, P. Carroll, J. Org. Chem. 1997, 62, 2555-2563; D. A. Cogan, G. C. Liu, K. J. Kim, B. J. Backes, J. A. Ellman, J. Am. Chem. Soc. 1998, 120, 8011-8019; D. A. Cogan, J. A. Ellman, J. Am. Chem. Soc. 1999, 121, 268-269; D. J. Weix, Y. L. Shi, J. A. Ellman, J. Am. Chem. Soc. 2005, 127, 1092-1093, X. Han, D. Krishnamurthy, P. Grover, Q. K. Fang, C. H. Senanayake, J. Am. Cheng. Soc. 2002, 124, 7880-7881; J. G. Ruano, I. Fernandez, M. del P. Catalina, A. A. Cruz, Tetrahedron Asymm., 1996, 7, 3407-3414; X. W. Sun, M. H. Xu, G. Q. Lin, Org. Lett. 2006, 8, 4979-4982; C. H. Zhao, L. Liu, D. Wang, Y. J. Chen, Eur. J. Org. Chem. 2006, 2977-2986; D. H. Hua, S. W. Miao, J. S. Chen, S. Iguchi, J. Org. Chem. 1991, 56, 4-6). For example, nucleophilic addition of boronates unto imines produces the essential precursor for the synthesis of bortezomib (Velcade), the first FDA approved proteasome inhibitor drug. Beenen, M. A.; An, C.; Ellman, J. A. J. Am. Chem. Soc. 2008, 130, 6910. Nucleophilic addition of cyanides unto imines produces natural and unnatural α-amino acids, and these are very useful building blocks for the synthesis of polypeptides. (R)-cetirizine dihydrochloride is used for the treatment of allergies, and it can be synthesized in high enantiopurity with the help of imine chemistry. Pflum, D. A.; Krishnamurthy, D.; Han, Z.; Wald, S. A.; Senanayake, C. H. Tetrahedron Lett. 2002, 43, 923. Aryl Grignard addition to imines is one of the key steps to synthesis of tubulin polymerization inhibitor (S)—N-acetylcolchinol. Besong, G.; Jarowicki, K.; Kocienski, P. J.; Sliwinski, E.; Boyle, F. T. Org. Biomol. Chem. 2006, 4, 2193. In addition, there are several pharmacologically and biologically important compounds containing amine functionalities, wherein imine chemistry provides easy access to synthesize key precursors, and as a result to achieve the synthesis of target molecules. Davis, F. A., Deng, J., Org. Lett. 2005, 7(4), 621; Pflurn, D. A. et al., Tetra. Lett. 43 (2002) 923.

With this interest, chiral N-phosphonylimines were developed and utilized in several asymmetric nucleophilic addition reactions such as aza-Henry, aza-Darzen, etc. to achieve excellent diastereoselectivities. A. Kattuboina, P. Kaur, T. Ai, G. Li, Chem. Biol. & Drug Design, 2008, 71, 216; A. Kattuboina, G. Li, Tetrahedron Lett. 2008, 49, 1573.

Later, research was conducted on further simplifying the imine chemistry by means of easy purification methods. In this process, the Group Assisted Purification (GAP) concept was developed, in which the addition reaction products are purified by simple washing with minimum amounts of solvents such as pentane, hexanes, heptane, ethyl acetate, etc. or mixture of solvents, depending on the solubility nature of the impurities and side products. Kaur, P.; Nguyen, T.; Li, G. Eur. J. Org. Chem. 2009, 912; Han, J.; Ai, T.; Li, G. Synthesis 2008, 16, 2519; Han, J.; Chen, Z.-X.; Ai, T.; Li, G. Chem. Biol. Drug Des. 2009,73, 203; Chen, Z.-X.; Ai, T.; Kaur, P.; Li, G. Tetrahedron Lett. 2009, 50, 1079; Ai, T.; Li, G. Bioorg. Med. Chem. Lett. 2009, 19, 3967; Kaur, P.; Shakya, G.; Sun, H.; Pan, Y.; Li, G. Org. Biomol. Chem. 2010, 8, 1091; Ai, T.; Han, J.; Chen, Z. X.; Li, G. Chem. Biol. Drug. Des. 2009, 73, 203; Kattuboina, A.; Kaur, P.; Ai, T.; Li, G. Chem. Biol. Drug Des. 2008, 71, 216; Ai, T.; Pindi, S.; Kattamuri, P. V.; Li, G. Sci. China Series B: Chem. 2010, 53, 125; Pindi, S.; Kaur, P.; Shakya, G.; Li, G. Chem. Biol. Drug Design, 2011, 75, 20.

### SUMMARY

To further explore this novel concept, new N-protection groups have been developed to avoid traditional column chromatography and assist the GAP technique, which is more economic and environmental friendly. As part of this discovery, novel chiral N-2-phenylpropyl-2-sulfinylimines were synthesized.

The present disclosure, therefore, provides the design and synthesis of novel chiral 2-arylpropyl-2-sulfinamides and corresponding imines, which can be utilized to synthesize chiral amines. Also provided herein is a new methodology to synthesize 2-arylpropyl-2-sulfonamides from the corresponding thiosulfinates, utilizing an alternative nitrogen source. The new methodologies provide successful synthesis of 2-arylpropyl-2-sulfonamides where the aryl group containing thiosulfinates fail to produce sulfonamides with the traditional Li/liquid NH₃ method.

In certain embodiments, provided herein is a compound according to Formula 1001a or 1001b:

[chemical structure]

wherein: Y is amino or imino; and R^{A} is lower alkyl or aryl.

In certain embodiments, provided herein is a compound according to Formula Ia or Ib:

[chemical structure]

wherein each R₁ is independently hydrogen, alkyl or aryl.

In certain embodiments, provided herein is a compound according to formula IIIa or IIIb:

[chemical structure]

wherein each of R₁, R₂, and R₃ is independently hydrogen, alkyl or aryl.

In certain embodiments, provided herein is a method for preparing a compound of Formula Ia, the method comprising: (a) reacting a compound of formula i2 in the presence of a compound of formula i3a to form a compound of formula i4a:

[chemical structure]

(b) reacting the compound of formula i4a in the presence of TBDMS-NH₂ to form a compound of formula i5a:

[chemical structure]

and (c) reacting the compound of formula i5a in the presence of TBAF to form the compound of formula Ia:

[chemical structure]

wherein each R₁ is independently hydrogen, alkyl or aryl.

In certain embodiments, provided herein is a method for preparing a compound of formula Ib, the method comprising: (a) reacting a compound of formula i2 in the presence of a compound of formula i3b to form a compound of formula i4b:

[chemical structure]

(b) reacting the compound of formula i4b in the presence of TBDMS-NH₂ to form a compound of formula i5b:

[chemical structure]

and (c) reacting the compound of formula i5b in the presence of TBAF to form the compound of formula Ib:

[chemical structure]

wherein each R₁ is independently hydrogen, alkyl or aryl.

In certain embodiments, provided herein is a method for preparing a compound of formula i2 from a compound of formula i1, the method comprising reacting a compound of formula i1 in the presence of an alkali halide, hydrogen peroxide and ethylacetate to form compound i2:

[chemical structure]

wherein each R₁ is independently hydrogen, alkyl or aryl.

In certain embodiments, provided herein is a method for preparing a compound of formula i5a, the method comprising: (a) reacting a compound of formula i2 in the presence of a compound of formula i3a to form a compound of formula i4a:

[chemical structure]

and (b) reacting the compound of formula i4a in the presence of TBDMS-NH₂ to form a compound of formula i5a:

[chemical structure]

wherein each R₁ is independently hydrogen, alkyl or aryl.

In certain embodiments, provided herein is a method for preparing a compound of formula i5b, the method comprising: (a) reacting a compound of formula i2 in the presence of a compound of formula i3b to form a compound of formula i4b:

[chemical structure]

and (b) reacting the compound of formula i4b in the presence of TBDMS-NH₂ to form a compound of formula i5b:

[chemical structure]

wherein each R₁ is independently hydrogen, alkyl or aryl.

In certain embodiments, provided here is a method for the preparation of a compound of formula IIIa, the method comprising reacting a compound of formula Ia with a compound of formula i6 to form a compound of formula IIIa:

[chemical structure]

wherein each of R₁, R₂, and R₃ is independently hydrogen, alkyl or aryl.

In certain embodiments, provided herein is a method for the preparation of a compound of formula IIIb, the method comprising reacting a compound of formula Ib with a compound of formula i6 to form a compound of formula IIIb:

[chemical structure]

wherein each of R₁, R₂, and R₃ is independently hydrogen, alkyl or aryl.

In certain embodiments, provided herein is a method for the preparation of a compound of formula 1002a, the method comprising reacting a compound of formula 1003a with a compound of formula i6 to form a compound of formula 1002a:

[chemical structure]

wherein each of R^{A}, R₂, and R₃ is independently hydrogen, alkyl or aryl.

The sulfinylimines described herein are useful, for example, in the chiral synthesis of Cetirizine using the method of Pflum et al. See, Pflum, D. A. et al., Tetra. Lett. 43 (2002) 923. The sulfinylimines described herein are also useful, for example, in the chiral synthesis of Agelastatin A. using the method of Davis and Deng. See, Davis, F. A., Deng, J., Org. Lett. 2005, 7(4), 621.

### DESCRIPTION OF EXEMPLARY EMBODIMENTS

Provided herein are novel chiral sulfinamide and imine compounds. Also provided herein are methods of synthesizing novel chiral sulfinamide and imine compounds comprising simplified purification methods when compared to prior methods. The novel chiral sulfinamide and imine compounds are useful, for example, in the synthesis of complex natural products and pharmaceutical important compounds.

### DEFINITIONS

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

The term “alkyl”, as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, refers to a saturated straight or branched hydrocarbon having one to six carbon atoms, i.e., C₁ to C₆ alkyl. In certain embodiments, the lower alkyl group is a primary, secondary, or tertiary hydrocarbon. The term includes both substituted and unsubstituted moieties.

The term “cycloalkyl”, as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms, i.e., C₃ to C₁₀ cycloalkyl. In some embodiments, the cycloalkyl has from 3 to 15 (C₃₋₁₅), from 3 to 10 (C₃₋₁₀), or from 3 to 7 (C₃₋₇) carbon atoms. In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “cycloalkenyl”, as used herein, unless otherwise specified, refers to an unsaturated cyclic hydrocarbon. In certain embodiments, cycloalkenyl refers to mono- or multicyclic ring systems that include at least one double bond. In certain embodiments, the cycloalkenyl group may be a bridged, non-bridged, and/or a fused bicyclic group. In certain embodiments, the cycloalkyl group includes three to ten carbon atoms, i.e., C₃ to C₁₀ cycloalkyl. In some embodiments, the cycloalkenyl has from 3 to 7 (C₃₋₁₀), or from 4 to 7 (C₃₋₇) carbon atoms.

“Alkylene” refers to divalent saturated aliphatic hydrocarbon groups particularly having from one to eleven carbon atoms which can be straight-chained or branched. In certain embodiments, the alkylene group contains 1 to 10 carbon atoms. The term includes both substituted and unsubstituted moieties. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” refers to monovalent olefinically unsaturated hydrocarbon groups, in certain embodiment, having up to about 11 carbon atoms, from 2 to 8 carbon atoms, or from 2 to 6 carbon atoms, which can be straight-chained or branched and having at least 1 or from 1 to 2 sites of olefinic unsaturation. The term includes both substituted and unsubstituted moieties. Exemplary alkenyl groups include ethenyl (i.e., vinyl, or —CH═CH₂), n-propenyl (—CH₂CH═CH₂), isopropenyl (—C(CH₃)═CH₂), and the like.

“Alkenylene” refers to divalent olefinically unsaturated hydrocarbon groups, in certain embodiments, having up to about 11 carbon atoms or from 2 to 6 carbon atoms which can be straight-chained or branched and having at least 1 or from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbon groups, in certain embodiments, having up to about 11 carbon atoms or from 2 to 6 carbon atoms which can be straight-chained or branched and having at least 1 or from 1 to 2 sites of alkynyl unsaturation. Non-limiting examples of alkynyl groups include acetylenic, ethynyl (—C□CH), propargyl (—CH₂C≡CH), and the like.

The term “aryl”, as used herein, and unless otherwise specified, refers to a cyclic aromatic hydrocarbon. In certain embodiments, aryl is furanyl, pyridinyl, phenyl, biphenyl, or naphthyl. The term includes both substituted and unsubstituted moieties. An aryl group can be substituted with any described moiety, including, but not limited to, one or more moieties selected from the group consisting of halogen (fluoro, chloro, bromo or iodo), alkyl, haloalkyl, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

“Alkoxy” refers to the group —OR′ where R′ is alkyl or cycloalkyl. Alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Amino” refers to the radical —NH₂.

“Carboxyl” or “carboxy” refers to the radical —C(O)OH.

The term “alkylamino” or “arylamino” refers to an amino group that has one or two alkyl or aryl substituents, respectively. In certain embodiments, the alkyl substituent is lower alkyl. In another embodiment, the alkyl or lower alkyl is unsubstituted.

“Halogen” or “halo” refers to chloro, bromo, fluoro or iodo.

“Monoalkylamino” refers to the group alkyl-NR′—, wherein R′ is selected from hydrogen and alkyl or cycloalkyl.

“Thioalkoxy” refers to the group —SR′ where R′ is alkyl or cycloalkyl.

The term “heterocyclyl” or “heterocyclic” refers to a monovalent monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Heterocyclyl groups are bonded to the rest of the molecule through the non-aromatic ring. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted as described herein.

The term “heteroaryl” refers to refers to a monovalent monocyclic aromatic group and/or multicyclic aromatic group that contain at least one aromatic ring, wherein at least one aromatic ring contains one or more heteroatoms independently selected from O, S, and N in the ring. Heteroaryl groups are bonded to the rest of the molecule through the aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein.

The term “alkylaryl” refers to an aryl group with an alkyl substituent. The term “aralkyl” or “arylalkyl” includes an alkyl group with an aryl substituent.

The term “alkylheterocyclyl” refers to a heterocyclyl group with an alkyl substituent. The term alkylheterocyclyl includes an alkyl group with a heterocyclyl substituent.

The term “alkylheteroaryl” refers to a heteroaryl group with an alkyl substituent. The term alkylheteroaryl includes an alkyl group with a heteroaryl substituent.

The term “protecting group” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “acyl” or “O-linked ester” refers to a group of the formula C(O)R′, wherein R′ is alkyl or cycloalkyl (including lower alkyl), carboxylate reside of amino acid, aryl including phenyl, alkaryl, arylalkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or arylalkyl sulphonyl including methanesulfonyl, the mono, di or triphosphate ester, trityl or monomethoxy-trityl, substituted benzyl, alkaryl, arylalkyl including benzyl, alkoxyalkyl including methoxymethyl, aryloxyalkyl such as phenoxymethyl. Aryl groups in the esters optimally comprise a phenyl group. In particular, acyl groups include acetyl, trifluoroacetyl, methylacetyl, cyclpropylacetyl, propionyl, butyryl, hexanoyl, heptanoyl, octanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoroheptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nonafluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxybenzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, 4-phenylbenzoyl.

The term “substantially free of” or “substantially in the absence of” with respect to a composition refers to a composition that includes at least 85% or 90% by weight, in certain embodiments 95%, 98%, 99% or 100% by weight, of a designated enantiomer of a compound. In certain embodiments, in the methods and compounds provided herein, the compounds are substantially free of enantiomers.

Similarly, the term “isolated” with respect to a composition refers to a composition that includes at least 85%, 90%, 95%, 98%, 99% or 100% by weight, of a compound, the remainder comprising other chemical species or enantiomers.

Similarly, the term “diastereomerically pure” with respect to a compound refers to a compound that includes at least 85% or 90% by weight, in certain embodiments 95%, 98%, 99% or 100% by weight of the designated diastereomer.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen”, the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “cycloalkyl,” “alkenyl,” “cycloalkenyl,” “alkynyl,” “aryl,” “alkoxy,” “alkoxycarbonyl,” “amino,” “carboxyl,” “alkylamino,” “arylamino,” “thioalkyoxy,” “heterocyclyl,” “heteroaryl,” “alkylheterocyclyl,” “alkylheteroaryl,” “acyl,” “aralkyl,” “alkaryl,” “purine,” “pyrimidine,” “carboxyl” and “amino acid” groups optionally comprise deuterium at one or more positions where hydrogen atoms are present, and wherein the deuterium composition of the atom or atoms is other than the natural isotopic composition.

Also as used herein, “alkyl,” “cycloalkyl,” “alkenyl,” “cycloalkenyl,” “alkynyl,” “aryl,” “alkoxy,” “alkoxycarbonyl,” “carboxyl,” “alkylamino,” “arylamino,” “thioalkyoxy,” “heterocyclyl,” “heteroaryl,” “alkylheterocyclyl,” “alkylheteroaryl,” “acyl,” “aralkyl,” “alkaryl,” “purine,” “pyrimidine,” “carboxyl” and “amino acid” groups optionally comprise carbon-13 at an amount other than the natural isotopic composition.

The term “protected”, as used herein and unless specified otherwise, refers to a group that is added to an oxygen, nitrogen or phosphorus atom to prevent its further reaction or for other purposes. A wide variety of oxygen, nitrogen and phosphorus protecting groups are known to those skilled in the art of organic synthesis.

Examples of suitable protecting groups include, but not limited to, benzoyl; substituted or unsubstituted alkyl groups, substituted or unsubstituted aryl groups, substituted or unsubstituted silyl groups; substituted or unsubstituted aromatic or aliphatic esters, such as, for example, aromatic groups like benzoyl, toluoyls (e.g. p-toluoyl), nitrobenzoyl, chlorobenzoyl; ether groups such as, for example, —C—O-aralkyl, —C—O-alkyl, or —C—O-aryl; and aliphatic groups like acyl or acetyl groups, including any substituted or unsubstituted aromatic or aliphatic acyl, —(C═O)-aralkyl, —(C═O)-alkyl, or —(C═O)-aryl; wherein the aromatic or aliphatic moiety of the acyl group can be straight-chained or branched; all of which may be further optionally substituted by groups not affected by the reactions comprising the improved synthesis (see Greene et al., Protective Groups in Organic Synthesis, John Wiley and Sons, 2nd Edition, 1991). For example, in one embodiment of the invention, the protecting groups are substituted by groups not affected by the reducing agent of choice, such as Red-Al. For the use of ethers as protective groups, attention is directed to U.S. Pat. No. 6,229,008 to Saischek et al., herein incorporated by reference, wherein it is reported that the use of an ether as a protective group may offer significant advantages for stability toward reagents and process conditions. This affords an ultimate advantage for separation, isolation, and purification of the desired product and thus, on the product's percent yield.

“Derivative,” “analog,” “chemical derivative,” “derivatizing,” and similar terms are given their ordinary meanings as well-known in the fields of chemistry, biochemistry, and/or biology. A derivative may be any chemical substance structurally related to another chemical substance and at least theoretically derivable from it. An analog may be a chemical or biological species that is similar enough to a parent species that it may substitute for the parent species in at least one set of chemical or biochemical interactions.

### Synthetic Methods

In certain embodiments, provided herein is a method for preparing a compound of Formula Ia, the method comprising: (a) reacting a compound of formula i2 in the presence of a compound of formula i3a to form a compound of formula i4a:

[chemical structure]

(b) reacting the compound of formula i4a in the presence of TBDMS-NH₂ to form a compound of formula i5a:

[chemical structure]

and (c) reacting the compound of formula i5a in the presence of TBAF to form the compound of formula Ia:

[chemical structure]

wherein each R₁ is independently hydrogen, alkyl or aryl. In certain embodiments, the reaction of step a proceeds substantially in the absence of compounds i3b and i4b. In certain embodiments, the reaction of step b proceeds substantially in the absence of compounds i4b and i5b. In certain embodiments, the reaction of step c proceeds substantially in the absence of compounds i5b and Ib.

In certain embodiments, provided herein is a method for preparing a compound of Formula Ib, the method comprising: (a) reacting a compound of formula i2 in the presence of a compound of formula i3b to form a compound of formula i4b:

[chemical structure]

(b) reacting the compound of formula i4b in the presence of TBDMS-NH₂ to form a compound of formula i5b:

[chemical structure]

and (c) reacting the compound of formula i5b in the presence of TBAF to form the compound of formula Ib:

[chemical structure]

wherein each R₁ is independently hydrogen, alkyl or aryl.

In certain embodiments, the reaction of step a proceeds substantially in the absence of compounds i3a and i4a. In certain embodiments, the reaction of step b proceeds substantially in the absence of compounds i4a and i5a. In certain embodiments, the reaction of step c proceeds substantially in the absence of compounds i5a and Ia.

In certain embodiments, a method for preparing a compound of formula i2 from a compound of formula i1 is provided, the method comprising reacting a compound of formula i1 in the presence of an alkali halide, hydrogen peroxide and ethylacetate to form compound i2:

[chemical structure]

wherein each R₁ is independently hydrogen, alkyl or aryl. In certain embodiments, the alkali halide is NaI.

In certain embodiments, a method for preparing a compound of formula i5a is provided, the method comprising: (a) reacting a compound of formula i2 in the presence of a compound of formula i3a to form a compound of formula i4a:

[chemical structure]

and (b) reacting the compound of formula i4a in the presence of TBDMS-NH₂ to form a compound of formula i5a:

[chemical structure]

wherein each R₁ is independently hydrogen, alkyl or aryl. In certain embodiments, the reaction of step a proceeds substantially in the absence of compounds i3b and i4b. In certain embodiments, the reaction of step b proceeds substantially in the absence of compounds i4b and i5b.

In certain embodiments, a method for preparing a compound of formula i5b is provided, the method comprising: (a) reacting a compound of formula i2 in the presence of a compound of formula i3b to form a compound of formula i4b:

[chemical structure]

and (b) reacting the compound of formula i4b in the presence of TBDMS-NH₂ to form a compound of formula i5b:

[chemical structure]

wherein each R₁ is independently hydrogen, alkyl or aryl. In certain embodiments, the reaction of step (a) proceeds substantially in the absence of compounds i3a and i4a. In certain embodiments, the reaction of step (b) proceeds substantially in the absence of compounds i4a and i5a.

In certain embodiments of the methods described herein, each R₁ is independently alkyl or aryl. In certain embodiments of the methods described herein, each R₁ is independently C₄-C₁₄ aryl. In certain embodiments of the methods described herein, each R₁ is independently C₄-C₁₀ aryl. In certain embodiments of the methods described herein, each R₁ is independently C₄-C₁₄ heteroaryl. In certain embodiments of the methods described herein, R₁ is independently C₄-C₁₀ heteroaryl. In certain embodiments of the methods described herein, each R₁ is independently furanyl, pyridinyl, phenyl, biphenyl, or naphthyl.

In certain embodiments of the methods described herein, each R₁ is independently lower alkyl. In certain embodiments of the methods described herein, each R₁ is independently C₁-C₂₀ alkyl. In certain embodiments of the methods described herein, each R₁ is independently C₁-C₁₀ alkyl. In certain embodiments of the methods described herein, each R₁ is independently C₁-C₅ alkyl.

In certain embodiments of the methods described herein, each R₁ is independently hydrogen, methyl, or isopropyl. In certain embodiments of the methods described herein, each R₁ is independently methyl or isopropyl. In certain embodiments of the methods described herein, wherein each R₁ is methyl. In certain embodiments of the methods described herein, each R₁ is isopropyl. In certain embodiments of the methods described herein, each R₁ is hydrogen.

In certain embodiments, provided herein is a method for the preparation of a compound of formula 1002a, the method comprising reacting a compound of formula 1003a with a compound of formula i6 to form a compound of formula 1002a:

[chemical structure]

wherein R^{A} is lower alkyl and each of R₂ and R₃ is independently hydrogen, alkyl or aryl. In certain embodiments, the reaction proceeds substantially in the absence of compound 1003b.

In certain embodiments, provided herein is a method for the preparation of a compound of formula 1002b, the method comprising reacting a compound of formula 1003b with a compound of formula i6 to form a compound of formula 1002b:

[chemical structure]

wherein R^{A} is lower alkyl and each of R₂ and R₃ is independently hydrogen, alkyl or aryl. In certain embodiments, the reaction proceeds substantially in the absence of compound 1003a.

In certain embodiments, provided herein is a method for the preparation of a compound of formula IIIa, the method comprising reacting a compound of formula Ia with a compound of formula i6 to form a compound of formula IIIa:

[chemical structure]

wherein each of R₁, R₂, and R₃ is independently hydrogen, alkyl or aryl. In certain embodiments of the above method, the reaction proceeds substantially in the absence of compound Ib.

In certain embodiments, provided herein is a method for the preparation of a compound of formula IIIb, the method comprising reacting a compound of formula Ib with a compound of formula i6 to form a compound of formula IIIb:

[chemical structure]

wherein each of R₁, R₂, and R₃ is independently hydrogen, alkyl or aryl. In certain embodiments of the above method, the reaction proceeds substantially in the absence of compound Ia.

In certain embodiments of the methods described above, each R₁ is independently alkyl or aryl. In certain embodiments of the methods described above, each R₁ is independently C₄-C₁₄ aryl. In certain embodiments of the methods described above, each R₁ is independently C₄-C₁₀ aryl. In certain embodiments of the methods described above, each R₁ is independently C₄-C₁₄ heteroaryl. In certain embodiments of the methods described above, each R₁ is independently C₄-C₁₀ heteroaryl. In certain embodiments of the methods described above, each R₁ is independently furanyl, pyridinyl, phenyl, biphenyl, or naphthyl.

In certain embodiments of the methods described above, each R₁ is independently lower alkyl. In certain embodiments of the methods described above, each R₁ is independently C₁-C₂₀ alkyl. In certain embodiments of the methods described above, each R₁ is independently C₁-C₁₀ alkyl. In certain embodiments of the methods described above, each R₁ is independently C₁-C₅ alkyl. In certain embodiments of the methods described above, each R₁ is independently hydrogen, methyl, or isopropyl. In certain embodiments of the methods described above, each R₁ is independently methyl or isopropyl. In certain embodiments of the methods described above, each R₁ is methyl. In certain embodiments of the methods described above, each R₁ is hydrogen. In certain embodiments of the methods described above, each R₁ is isopropyl.

In certain embodiments of the methods described above, each R₂ is independently alkyl or aryl. In certain embodiments of the methods described above, each R₂ is independently C₄-C₁₄ aryl. In certain embodiments of the methods described above, each R₂ is independently C₄-C₁₀ aryl. In certain embodiments of the methods described above, each R₂ is independently C₄-C₁₄ heteroaryl. In certain embodiments of the methods described above, each R₂ is independently C₄-C₁₀ heteroaryl. In certain embodiments of the methods described above, each R₂ is independently furanyl, pyridinyl, phenyl, biphenyl, or naphthyl.

In certain embodiments of the methods described above, each R₂ is independently lower alkyl. In certain embodiments of the methods described above, each R₂ is independently C₁-C₂₀ alkyl. In certain embodiments of the methods described above, each R₂ is independently C₁-C₁₀ alkyl. In certain embodiments of the methods described above, each R₂ is independently C₁-C₅ alkyl. In certain embodiments of the methods described above, each R₂ is independently hydrogen, methyl, or isopropyl. In certain embodiments of the methods described above, each R₂ is independently methyl or isopropyl. In certain embodiments of the methods described above, each R₂ is methyl. In certain embodiments of the methods described above, each R₂ is hydrogen. In certain embodiments of the methods described above, each R₂ is isopropyl.

In certain embodiments of the methods described above, each R₃ is independently alkyl or aryl. In certain embodiments of the methods described above, each R₃ is independently C₄-C₁₄ aryl. In certain embodiments of the methods described above, each R₃ is independently C₄-C₁₀ aryl. In certain embodiments of the methods described above, each R₃ is independently C₄-C₁₄ heteroaryl. In certain embodiments of the methods described above, each R₃ is independently C₄-C₁₀ heteroaryl. In certain embodiments of the methods described above, each R₃ is independently furanyl, pyridinyl, phenyl, biphenyl, or naphthyl.

In certain embodiments of the methods described above, each R₃ is independently lower alkyl. In certain embodiments of the methods described above, each R₃ is independently C₁-C₂₀ alkyl. In certain embodiments of the methods described above, each R₃ is independently C₁-C₁₀ alkyl. In certain embodiments of the methods described above, each R₃ is independently C₁-C₅ alkyl. In certain embodiments of the methods described above, each R₃ is independently hydrogen, methyl, or isopropyl. In certain embodiments of the methods described above, each R₃ is independently methyl or isopropyl. In certain embodiments of the methods described above, each R₃ is methyl. In certain embodiments of the methods described above, each R₃ is hydrogen. In certain embodiments of the methods described above, each R₃ is isopropyl. In certain embodiments of the methods described above, each R₃ is cycloalkyl. In certain embodiments of the methods described above, each R₃ is C₃-C₁₄ cycloalkyl. In certain embodiments of the methods described above, each R₃ is C₃-C₈ cycloalkyl.

In certain embodiments of the methods described above, each R₃ is independently hydrogen, methyl, or isopropyl. In certain embodiments of the methods described above, each R₃ is independently methyl or isopropyl. In certain embodiments of the methods described above, each R₃ is methyl. In certain embodiments of the methods described above, each R₃ is isopropyl. In certain embodiments of the methods described above, R₃ is hydrogen or methyl. In certain embodiments of the methods described above, R₃ is hydrogen. In certain embodiments of the methods described above, R₃ is methyl.

In certain embodiments of the methods described above, R₂ is phenyl, 1-napthyl, 2-methyl-phenyl, 4-methyl-phenyl, trans-Ph-CH═CH, 2-furyl, isobutyl, cyclohexyl, or —C(O)OCH₂CH₃.

In certain embodiments of the methods described above: R₁ is hydrogen, alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, alkyl or aryl. In certain embodiments of the methods described above: R₁ is hydrogen, lower alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, lower alkyl or aryl. In certain embodiments of the methods described above: R₁ is alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, alkyl or aryl. In certain embodiments of the methods described above: R₁ is hydrogen, lower alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is lower alkyl or aryl. In certain embodiments of the methods described above: R₁ is hydrogen, alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is lower alkyl or aryl. In certain embodiments of the methods described above: R₁ is hydrogen, lower alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is lower alkyl or aryl.

In certain embodiments of the methods described above: R₁ is hydrogen; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen or methyl.

### Compounds

In certain embodiments, provided herein is a compound according to Formula 1001a or 1001b:

[chemical structure]

wherein: Y is amino or imino; and R^{A} is lower alkyl or aryl.

In certain embodiments, provided herein is a compound according to Formula 1002a or 1002b:

[chemical structure]

wherein: R^{A} is as described in the context of Formula 1001; each R₂ is independently lower alkyl, aryl, alkylaryl, arylalkenyl, alkoxycarbonyl, nitroaryl, or cycloalkyl; and each R₃ is independently hydrogen or lower alkyl.

In certain embodiments, provided herein is a compound according to Formula Ia or Ib:

[chemical structure]

wherein each R₁ is independently hydrogen, alkyl or aryl.

In certain embodiments, provided herein is a compound according to Formula IIa or IIb:

[chemical structure]

wherein R₁ is as defined in the context of formulas Ia and Ib.

In certain embodiments, provided herein is a substantially diastereomerically pure compound according to formula 1002a, 1002b, IIIa or IIIb:

[chemical structure]

wherein: R^{A} is as described in the context of Formula 1001; each of R₁ is independently hydrogen, alkyl or aryl; each R₂ is independently lower alkyl, aryl, alkylaryl, arylalkenyl, alkoxycarbonyl, nitroaryl, or cycloalkyl; and each R₃ is independently hydrogen or lower alkyl.

In certain embodiments, provided herein is a substantially diastereomerically pure compound according to any of Formulas Ia, Ib, IIa, or IIb:

[chemical structure]

wherein each R₁ is independently hydrogen, alkyl or aryl. In an embodiment, provided herein are compositions comprising a compound of formula Ia which does not comprise a compound of formula Ib. In an embodiment, provided herein are compositions comprising a compound of formula Ib which does not comprise a compound of formula Ia. In an embodiment, provided herein are compositions comprising a compound of formula IIa which does not comprise a compound of formula IIb. In an embodiment, provided herein are compositions comprising a compound of formula IIb which does not comprise a compound of formula IIa.

In an embodiment, a compound of any of formulas Ia, Ib, IIa, or IIb is provided wherein each R₁ is independently alkyl or aryl. In an embodiment, a compound of any of formulas Ia, Ib, IIa, or IIb is provided wherein each R₁ is independently C₄-C₁₄ aryl. In an embodiment, a compound of any of formulas IIa, Ib, IIa, or IIb is provided wherein each R₁ is independently C₄-C₁₀ aryl. In an embodiment, a compound of any of formulas Ia, Ib, IIa, or IIb is provided wherein each R₁ is independently C₄-C₁₄ heteroaryl. In an embodiment, a compound of any of formulas Ia, Ib, IIa, or IIb is provided wherein each R₁ is independently C₄-C₁₀ heteroaryl. In an embodiment, a compound of any of formulas Ia, Ib, IIa, or IIb is provided wherein each R₁ is independently furanyl, pyridinyl, phenyl, biphenyl, or naphthyl.

In an embodiment, a compound of any of formulas Ia, Ib, IIa, or IIb is provided wherein each R₁ is independently lower alkyl. In an embodiment, a compound of any of formulas Ia, Ib, IIa, or IIb is provided wherein each R₁ is independently C₁-C₂₀ alkyl. In an embodiment, a compound of any of formulas Ia, Ib, IIa, or IIb is provided wherein each R₁ is independently C₁-C₁₀ alkyl. In an embodiment, a compound of any of formulas Ia, Ib, IIa, or IIb is provided wherein each R₁ is independently C₁-C₅ alkyl.

In an embodiment, a compound of any of formulas Ia, Ib, IIa, or IIb is provided wherein each R₁ is independently hydrogen, methyl, or isopropyl. In an embodiment, a compound of any of formulas Ia, Ib, IIa, or IIb is provided wherein each R₁ is independently methyl or isopropyl. In an embodiment, a compound of any of formulas Ia, Ib, IIa, or IIb is provided wherein each R₁ is methyl. In an embodiment, a compound of any of formulas Ia, Ib, IIa, or IIb is provided wherein each R₁ is isopropyl. In an embodiment, a compound of any of formulas Ia, Ib, IIa, or IIb is provided wherein each R₁ is hydrogen.

In certain embodiments, provided herein is a compound according to formula IIIa or IIIb:

[chemical structure]

wherein each of R₁, R₂, and R₃ is independently hydrogen, alkyl or aryl.

In certain embodiments, provided herein is a substantially diastereomerically pure compound according to formula IIIa or IIIb:

[chemical structure]

wherein each of R₁, R₂, and R₃ is independently hydrogen, alkyl or aryl. In an embodiment, provided herein are compositions comprising a compound of formula IIIa which does not comprise a compound of formula IIIb. In an embodiment, provided herein are compositions comprising a compound of formula IIIb which does not comprise a compound of formula IIIa.

In an embodiment, a compound of any of formulas IIIa or IIIb is provided wherein each R₁ is independently alkyl or aryl. In an embodiment, a compound of any of formulas IIIa or IIIb is provided wherein each R₁ is independently C₄-C₁₄ aryl. In an embodiment, a compound of any of formulas IIIa or IIIb is provided wherein each R₁ is independently C₄-C₁₀ aryl. In an embodiment, a compound of any of formulas IIIa or IIIb is provided wherein each R₁ is independently C₄-C₁₄ heteroaryl. In an embodiment, a compound of any of formulas IIIa or IIIb is provided wherein each R₁ is independently C₄-C₁₀ heteroaryl. In an embodiment, a compound of any of formulas IIIa or IIIb is provided wherein each R₁ is independently furanyl, pyridinyl, phenyl, biphenyl, or naphthyl.

In an embodiment, a compound of any of formulas IIIa or 111b is provided wherein each R₁ is independently lower alkyl. In an embodiment, a compound of any of formulas IIIa or IIIb is provided wherein each R₁ is independently C₁-C₂₀ alkyl. In an embodiment, a compound of any of formulas IIIa or IIIb is provided wherein each R₁ is independently C₁-C₁₀ alkyl. In an embodiment, a compound of any of formulas IIIa or IIIb is provided wherein each R₁ is independently C₁-C₅ alkyl. In an embodiment, a compound of any of formulas IIIa or IIIb is provided wherein each R₁ is independently hydrogen, methyl, or isopropyl. In an embodiment, a compound of any of formulas IIIa or IIIb is provided wherein each R₁ is independently methyl or isopropyl. In an embodiment, a compound of any of formulas IIIa or IIIb is provided wherein each R₁ is methyl. In an embodiment, a compound of any of formulas IIIa or IIIb is provided wherein each R₁ is hydrogen. In an embodiment, a compound of any of formulas IIIa or IIIb is provided wherein each R₁ is isopropyl.

In an embodiment, a compound of any of formulas IIIa or IIIb is provided wherein each R₂ is independently alkyl or aryl. In an embodiment, a compound of any of formulas IIIa or IIIb is provided wherein each R₂ is independently C₄-C₁₄ aryl. In an embodiment, a compound of any of formulas IIIa or IIIb is provided wherein each R₂ is independently C₄-C₁₀ aryl. In an embodiment, a compound of any of formulas IIIa or IIIb is provided wherein each R₂ is independently C₄-C₁₄ heteroaryl. In an embodiment, a compound of any of formulas IIIa or IIIb is provided wherein each R₂ is independently C₄-C₁₀ heteroaryl. In an embodiment, a compound of any of formulas IIIa or IIIb is provided wherein each R₂ is independently furanyl, pyridinyl, phenyl, biphenyl, or naphthyl.

In an embodiment, a compound of any of formulas IIIa or IIIb is provided wherein each R₂ is independently lower alkyl. In an embodiment, a compound of any of formulas IIIa or IIIb is provided wherein each R₂ is independently C₁-C₂₀ alkyl. In an embodiment, a compound of any of formulas IIIa or IIIb is provided wherein each R₂ is independently C₁-C₁₀ alkyl. In an embodiment, a compound of any of formulas IIIa or IIIb is provided wherein each R₂ is independently C₁-C₅ alkyl. In an embodiment, a compound of any of formulas IIIa or IIIb is provided wherein each R₂ is independently hydrogen, methyl, or isopropyl. In an embodiment, a compound of any of formulas IIIa or IIIb is provided wherein each R₂ is independently methyl or isopropyl. In an embodiment, a compound of any of formulas IIIa or IIIb is provided wherein each R₂ is methyl. In an embodiment, a compound of any of formulas IIIa or IIIb is provided wherein each R₂ is hydrogen. In an embodiment, a compound of any of formulas IIIa or IIIb is provided wherein each R₂ is isopropyl.

In an embodiment, a compound of any of formulas IIIa or IIIb is provided wherein each R₃ is independently alkyl or aryl. In an embodiment, a compound of any of formulas IIIa or IIIb is provided wherein each R₃ is independently C₄-C₁₄ aryl. In an embodiment, a compound of any of formulas IIIa or IIIb is provided wherein each R₃ is independently C₄-C₁₀ aryl. In an embodiment, a compound of any of formulas IIIa or IIIb is provided wherein each R₃ is independently C₄-C₁₄ heteroaryl. In an embodiment, a compound of any of formulas IIIa or IIIb is provided wherein each R₃ is independently C₄-C₁₀ heteroaryl. In an embodiment, a compound of any of formulas IIIa or IIIb is provided wherein each R₃ is independently furanyl, pyridinyl, phenyl, biphenyl, or naphthyl.

In an embodiment, a compound of any of formulas IIIa or IIIb is provided wherein each R₃ is independently lower alkyl. In an embodiment, a compound of any of formulas IIIa or IIIb is provided wherein each R₃ is independently C₁-C₂₀ alkyl. In an embodiment, a compound of any of formulas IIIa or IIIb is provided wherein each R₃ is independently C₁-C₁₀ alkyl. In an embodiment, a compound of any of formulas IIIa or IIIb is provided wherein each R₃ is independently C₁-C₅ alkyl. In an embodiment, a compound of any of formulas IIIa or IIIb is provided wherein each R₃ is independently hydrogen, methyl, or isopropyl. In an embodiment, a compound of any of formulas IIIa or IIIb is provided wherein each R₃ is independently methyl or isopropyl. In an embodiment, a compound of any of formulas IIIa or IIIb is provided wherein each R₃ is methyl. In an embodiment, a compound of any of formulas IIIa or IIIb is provided wherein each R₃ is hydrogen. In an embodiment, a compound of any of formulas IIIa or IIIb is provided wherein each R₃ is isopropyl. In an embodiment, a compound of any of formulas IIIa or IIIb is provided wherein each R₃ is cycloalkyl. In an embodiment, a compound of any of formulas IIIa or IIIb is provided wherein each R₃ is C₃-C₁₄ cycloalkyl. In an embodiment, a compound of any of formulas IIIa or IIIb is provided wherein each R₃ is C₃-C₈ cycloalkyl.

In an embodiment, a compound of any of formulas IIIa or IIIb is provided wherein each R₃ is independently hydrogen, methyl, or isopropyl. In an embodiment, a compound of any of formulas IIIa or IIIb is provided wherein each R₃ is independently methyl or isopropyl. In an embodiment, a compound of any of formulas IIIa or IIIb is provided wherein each R₃ is methyl. In an embodiment, a compound of any of formulas IIIa or IIIb is provided wherein each R₃ is isopropyl. In an embodiment, a compound of any of formulas IIIa or IIIb is provided wherein R₃ is hydrogen or methyl. In an embodiment, a compound of any of formulas IIIa or IIIb is provided wherein R₃ is hydrogen. In an embodiment, a compound of any of formulas IIIa or IIIb is provided wherein R₃ is methyl.

In an embodiment, a compound of any of formulas IIIa or IIIb is provided wherein R₂ is phenyl, 1-napthyl, 2-methyl-phenyl, 4-methyl-phenyl, trans-Ph-CH═CH, 2-furyl, isobutyl, cyclohexyl, or —C(O)OCH₂CH₃.

In an embodiment, a compound of any of formulas IIIa or IIIb is provided wherein: R₁ is hydrogen, alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, alkyl or aryl. In an embodiment, a compound of any of formulas IIIa or IIIb is provided wherein: R₁ is hydrogen, lower alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, lower alkyl or aryl. In an embodiment, a compound of any of formulas IIIa or IIIb is provided wherein: R₁ is alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen, alkyl or aryl. In an embodiment, a compound of any of formulas IIIa or IIIb is provided wherein: R₁ is hydrogen, lower alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is lower alkyl or aryl. In an embodiment, a compound of any of formulas IIIa or IIIb is provided wherein: R₁ is hydrogen, alkyl or aryl; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is lower alkyl or aryl. In an embodiment, a compound of any of formulas IIIa or IIIb is provided wherein: R₁ is hydrogen, lower alkyl or aryl; R₂ is phenyl, 1-napthyl, 2-methyl-phenyl, 4-methyl-phenyl, trans-Ph-CH═CH—, 2-furl, isobutyl, cyclohexyl, or —C(O)OCH₂CH₃; and R₃ is lower alkyl or aryl.

In an embodiment, a compound of any of formulas IIIa or IIIb is provided wherein: R₁ is hydrogen; R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and R₃ is hydrogen or methyl.

In some embodiments, provided herein is processes for the preparation of compounds as described herein, e.g., of Formula 1001a, 1001b, 1002a, 1002b, 1003a, 1003b, Ia, Ib, IIa, IIb, IIIa, or IIIb, as described in more detail elsewhere herein.

Optically Active Compounds

It is appreciated that compounds provided herein have several chiral centers and are prepared or isolated in optically active forms, for example diastereomerically pure forms. Some compounds may exhibit polymorphism. It is well known in the art how to prepare optically active forms of the diastereomerically pure compounds provided herein, for example, by resolution of the racemic form by recrystallization techniques, by synthesis from optically-active starting materials, by chiral synthesis, or by chromatographic separation using a chiral stationary phase, such as high-performance liquid chromatography.

Diastereomerically pure sulfinamide and imine compounds and purified compositions comprising the diastereomerically pure sulfinamide and imine compounds can be prepared according to techniques known to those of skill in the art. Examples of methods to obtain diastereomerically pure materials are known in the art, and include at least the following:
a) physical separation of crystals—a technique whereby macroscopic crystals of the individual enantiomers are manually separated. This technique can be used if crystals of the separate enantiomers exist, i.e., the material is a conglomerate, and the crystals are visually distinct;
b) simultaneous crystallization—a technique whereby the individual enantiomers are separately crystallized from a solution of the racemate, possible only if the latter is a conglomerate in the solid state;
c) enzymatic resolutions—a technique whereby partial or complete separation of a racemate by virtue of differing rates of reaction for the enantiomers with an enzyme;
d) enzymatic asymmetric synthesis—a synthetic technique whereby at least one step of the synthesis uses an enzymatic reaction to obtain an enantiomerically pure or enriched synthetic precursor of the desired enantiomer;
e) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
f) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
g) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
h) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
i) enantiospecific synthesis from non-racemic precursors—a synthetic technique whereby the desired enantiomer is obtained from non-chiral starting materials and where the stereochemical integrity is not or is only minimally compromised over the course of the synthesis;
j) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
k) chiral gas chromatography—a technique whereby the racemate is volatilized and enantiomers are separated by virtue of their differing interactions in the gaseous mobile phase with a column containing a fixed non-racemic chiral adsorbent phase;
l) extraction with chiral solvents—a technique whereby the enantiomers are separated by virtue of preferential dissolution of one enantiomer into a particular chiral solvent;
m) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In some embodiments, provided are compositions of diastereomerically pure sulfinamide and imine compounds that are substantially free of a designated diastereomer of that compound. In certain embodiments, in the methods and compounds of this invention, the compounds are substantially free of other diastereomers. In some embodiments, a composition includes a compound that is at least 85%, 90%, 95%, 98%, 99% or 100% by weight, of the compound, the remainder comprising other chemical species or diastereomers.

### Isotopically Enriched Compounds

Also provided herein are isotopically enriched compounds, including but not limited to isotopically enriched diastereomerically pure sulfinamide and imine compounds.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

### Preparation of Compounds

The compounds provided herein can be prepared, isolated or obtained by any method apparent to those of skill in the art. Exemplary methods of preparation are described in detail in the examples below. In certain embodiments, compounds provided herein can be prepared according to Exemplary Preparation Schemes 1a and 1b, as discussed further below.

[chemical structure]

[chemical structure]

In certain embodiments, one or more protection or deprotection steps may be included in the methods of preparation described in Exemplary Preparation Schemes 1a and 1b. In certain embodiments, provided herein is a compound prepared according to the above Exemplary Preparation Scheme 1a or 1b.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hrs (hours); min (minutes); MS (mass spectrometry); ESI (electrospray ionization); TLC (thin layer chromatography); HPLC (high pressure liquid chromatography or high performance liquid chromatography); THF (tetrahydrofuran); CDCl₃ (deuterated chloroform); AcOH (acetic acid); DCM (dichloromethane); DMSO (dimethylsulfoxide); DMSO-d₆ (deuterated dimethylsulfoxide); EtOAc (ethyl acetate); MeOH (methanol); and BOC (t-butyloxycarbonyl).

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

The Examples below provide methods of synthesis of (R_{S})-2-phenylpropyl-2-sulfinamides and (S_{S})-2-phenylpropyl-2-sulfinamides. Also provided are methods of synthesis of other sulfinamides, such as (R_{S})-2-arylpropyl-2-sulfinamides. In addition, provided are methods to synthesize sulfonamides from thiosulfinates, wherein the thiosulfinates have aromatic groups. Also provided are methods using TBDMS-NH₂ as a nitrogen source to provide chiral sulfinamides from chiral thiosulfinates without racemization or side products. Also provided are methods for the preparation of (R_{S})-2-phenylpropyl-2-sulfinylimines from free sulfinamides.

### ABBREVIATIONS

TBDMS is tert-butyldimethylsilyl. TBAF is tetra-n-butylammonium fluoride. HPLC is high-performance liquid chromatography. THF is tetrahydrofuran. Ph is phenyl. M.S. is molecular sieve.

### Example 1

### Synthesis of (R_{S})-2-phenylpropyl-2-sulfinamide

[chemical structure]

(R_{S})-2-phenylpropyl-2-sulfinamides were synthesized starting from thiol 1. As shown in the scheme 1, thiol 1 was converted into disulfide 2, which was then oxidized in presence of commercially available chiral ligand (1S,2R)-3a to produce (R_{S})-thiosulfinate (R_{S})-4a. The chiral thiosulfinate (R_{S})-4-a was converted to TBDMS protected sulfinamide (R_{S})-5a with TBDMS-NH₂. The final deprotection of TBDMS yielded the desired (R_{S})-2-phenylpropyl-2-sulfinamide (R_{S})-D1 with good overall yields.

1.1. Synthesis of Disulfide 2

To a stirred solution of thiol 1 (78.8 mmol) in 240 mL of ethyl acetate was added NaI (236 mg, 1.57 mmol), and to this reaction mixture 30% aq. H₂O₂ (8.9 mL, 78.8 mmol) was added slowly at room temperature. Reaction mixture was stirred at room temperature for 18 hours. At this stage, saturated aq. Na₂S₂O₃ was added, and the mixture was extracted with ethyl acetate. The resulting organic layer was dried over anhydrous Na₂SO₄ and evaporated under reduced pressure. The crude mixture was purified by column chromatography with pure hexanes as eluant to give 9.29 g (78%) of disulfide 2.

¹H NMR (CDCl₃, 400 MHz): δ=1.75 (s, 12H), 7.42-7.44 (m, 2H), 7.49-7.54 (m, 4H), 7.67-7.69 (m, 4H). ¹³C NMR (CDCl₃, 100 MHz): δ=29.5, 52.2, 127.1, 127.4, 128.4, 128.5, 145.9.

1.2. Synthesis of Thiosulfinate (R_{S})-4-a

A 100 mL Schlenk flask was loaded with ligand (S_{S})-3a (62.8 mg, 0.1719 mmol) and vanadyl acetylacetanoate (44 mg, 0.165 mmol). Acetone (20 mL) was added and stirred the resulting dark-green solution at room temperature for 30 minutes, while open to the air. To this solution, disulfide 2 (10 g, 33.05 mmol) was added. The resulting mixture was cooled to 0° C. and 30% aq. H₂O₂ was added slowly with syringe pump over 20 hours. The dark-brown color solution was stirred for another 26 hours at 0° C. The reaction was quenched with saturated aq. Na₂S₂O₃, the resulting mixture was extracted with ethyl acetate. The combined organic layers were washed with brine and dried over anhydrous Na₂SO₄. The organic layer was evaporated under reduced pressure to afford crude (R_{S})-thiosulfinate (R_{S})-4-a as white solid, which was washed with hexanes to provide 7.9 g (75%) of pure (R_{S})-thiosulfinate (R_{S})-4-a with 85% ee. The enantiomeric excess was further improved to 94% by washing with hexanes (10 mL of hexanes required for 1 g of thiosulfinate). HPLC, Daicel ChiralPak AS-H column, 98:2 Hexanes/ⁱPrOH; 1.0 mL/min, 230 nm, t_{R}=14.7 min; t_{S}=19.1 min; [α]_{D}²⁵=+126.2° (c=1.0, CHCl₃); ¹H NMR (CDCl₃, 400 MHz): δ=1.63 (s, 3H), 1.77-1.82 (m, 9H), 7.24-7.41 (m, 1H). ¹³C NMR (CDCl₃, 100 MHz): δ=21.8, 24.4, 31.0, 31.9, 53.3, 65.5, 126.6, 126.7, 127.4, 128.1, 128.3, 128.4, 139.8, 145.1.

1.3. Synthesis of TBDMS Protected Sulfinamide (R_{S})-5a

TBDMS-NH₂ was prepared from TBDMS-Cl by a known literature method. Urgaonkar, S.; Cortese, J. F.; Barker, R. H.; Cormwell, M.; Serrano, A. E.; Wirth, D. F.; Clardy, J.; Mazitschek, R. Org. Lett. 2010, 12, 3998. Freshly synthesized 1M solution of TBDMS-NH₂ (35.1 mL, 35.1 mmol) in THF was taken in a dry round bottomed flask under argon. To this solution, 1.6 M n-BuLi (22.0 mL, 35.16 mmol) was added drop wise at −78° C., and the mixture was stirred at the same temperature. After 30 minutes the pre dissolved solution of thiosulfinate (R_{S})-4-a (2.8 g, 8.79 mmol) in THF was added slowly at −78° C. The reaction temperature raised to room temperature slowly, and stirred until the starting material consumed, monitored by TLC. At this stage, reaction quenched with 1 mL aq. NR₄Cl solution, and extracted with ethyl acetate. The organic layer was dried over anhydrous Na₂SO₄, and evaporated under reduced pressure to afford crude TBDMS protected sulfinamide (R_{S})-5a. The crude product was purified by column chromatography to get 2.45 g (93%) of compound (R_{S})-5a as white solid with 94% ee. HPLC, AS-H column, 93:7 Hexanes/ⁱPrOH; 1.0 mL/min, 254 nm, t_{S}=4.8 min; t_{R}=7.2 min; [α]_{D}²⁵=−31.8° (c=1.0, CHCl₃); ¹H NMR (CDCl₃, 400 MHz): δ=0.02 (d, J=10.1 Hz, 6H), 0.76 (s, 9H), 1.52 (s, 3H), 1.65 (s, 3H), 2.48 (br, 1H), 7.28-7.34 (m, 1H), 7.35-7.40 (m, 3H). ¹³C NMR (CDCl₃, 100 MHz): δ=−4.7, −4.2, 17.5, 22.0, 22.4, 25.5, 62.8, 127.7, 127.8, 128.2, 136.7.

1.4. Synthesis of Sulfinamide (R_{S})-D1

A dry round bottom flask was charged with TBDMS protected sulfinamide (R_{S})-5a (1.32 g, 4.44), and THF (23 mL) under argon and was added TBAF (5.33 mL, 5.33 mmol) at 0° C. After 1 hour, TLC indicated that all starting material was consumed. Then the reaction was quenched with 1 mL of water and extracted with ethyl acetate. The organic layer was dried over anhydrous Na₂SO₄, and evaporated under reduced pressure to afford crude sulfinamide (R_{S})-D1. The crude product was purified by column chromatography with ethyl acetate as eluant to afford pure sulfinamide (R_{S})-D1 0.8 g (98%) with 94% ee. HPLC, OD-H column, 93:7 Hexanes/ⁱPrOH; 1.0 mL/min, 254 nm, t_{R}=21.0 min; t_{S}=24.9 min; [α]_{D}²⁵=−100.2° (c=1.0, CHCl₃); ¹H NMR (CDCl₃, 400 MHz): δ=1.52 (s, 3H), 1.60 (s, 3H), 3.69 (br, 2H) 7.24-7.27 (m, 1H), 7.31-7.39 (m, 4H). ¹³C NMR (CDCl₃, 100 MHz): δ=21.7, 22.5, 61.0, 127.3, 127.5, 127.7, 136.5.

1.5. Synthesis of (S_{S}) Sulfinamides (S_{S})-D1

[chemical structure]

As shown in Scheme 4, using organocatalysis, commercially available ligand (1R,2S)-3a ((1R,2S)-1-[(3,5-Di-tert-butyl-2-hydroxybenzylidene)amino]-2-indanol) produces the (S_{S})-thiosulfinate (S_{S})-4-a, which is an enantiomer of (R_{S})-4-a. Subsequently, (S_{S})-sulfinamides (S_{S})-D1 and their imines are synthesized using the same protocols shown in Scheme 1 and Scheme 3.

Absolute stereochemistry was determined from the X-ray structure of the nucleophilic addition product, not shown. Stereochemistry of the products was confirmed from the rotation value. Stereochemical purity was determined by HPLC.

### Example 2

General procedure for the synthesis of (R_{S})-2-phenylpropyl-2-sulfinylimines

[chemical structure]

[chemical structure]

To a stirred solution of sulfinamide (R_{S})-D1 (150 mg, 0.818 mmol), in freshly distilled dichloromethane (20 mL), was added respective aldehyde (1.636 mmol), Ti(OEt)₄ (1.22 mL, 5.81 mmol) under argon. The flask was fitted with condenser and refluxed the reaction for 12-16 hours. Then the reaction was quenched with water (5 mL), and the precipitate was filtered through celite pad. The filter cake was washed with dichloromethane, and the filtrate was extracted with the same. The organic layer was dried over anhydrous Na₂SO₄, and evaporated under reduced pressure to afford crude sulfinylimine. The crude product was purified by column chromatography and hexanes, ethyl acetate mixture (4:1) was used as an eluant mixture to get corresponding sulfinylimines. In case of glyoxylate 6i, for the synthesis of its imine M.S. (4 Å) was used instead of Ti(OEt)₄. Results are provided in Table 1.

Absolute stereochemistry was determined from the X-ray structure of the nucleophilic addition product, not shown. Stereochemistry of the products was confirmed from the rotation value. Stereochemical purity was determined by HPLC.

Compound (R_{S})-7a: [α]_{D}²⁵=+17.7° (c=1.0, CHCl₃); ¹H NMR (CDCl₃, 400 MHz): δ=1.69 (s, 3H), 1.75 (s, 3H), 7.22-7.31 (m, 3H), 7.37-7.46 (m, 5H), 7.65-7.68 (m, 2H), 8.29 (s, 1H). ¹³C NMR (CDCl₃, 100 MHz): δ=21.1, 21.8, 64.4, 127.3, 127.5, 127.8, 128.7, 129.1, 132.2, 133.8, 138.1, 162.6.

Compound (R_{S})-7b: [α]_{D}²⁵=+34.5° (c=1.0, CHCl₃); ¹H NMR (CDCl₃, 400 MHz): δ=1.68 (s, 3H), 1.82 (s, 3H), 7.22-7.35 (m, 3H), 7.45-7.53 (m, 5H), 7.82-7.86 (m, 2H), 7.96 (d, J=8.2 Hz, 1H), 8.70-8.73 (m, 1H), 8.82 (s, 1H). ¹³C NMR (CDCl₃, 100 MHz): δ=19.9, 22.6, 64.4, 124.7, 125.0, 126.3, 127.3, 127.6, 127.8, 128.0, 128.6, 129.1, 130.9, 132.5, 133.3, 133.7, 138.6, 162.8.

Compound (R_{S})-7c: [α]_{D}²⁵=−53.9° (c=1.0, CHCl₃); ¹H NMR (CDCl₃, 400 MHz): δ=1.68 (s, 3H), 1.74 (s, 3H), 2.37 (s, 3H), 7.15-7.17 (m, 2H), 7.21-7.24 (m, 2H), 7.27-7.33 (m, 3H), 7.37-7.40 (m, 2H), 7.71-7.73 (m, 1H), 8.49 (s, 1H). ¹³C NMR (CDCl₃, 100 MHz): δ=19.6, 21.2, 21.9, 64.3, 126.1, 127.4, 127.5, 127.8, 129.2, 131.1, 131.9, 161.6.

Compound (R_{S})-7d: [α]_{D}²⁵=+58.3° (c=1.0, CHCl₃); ¹H NMR (CDCl₃, 400 MHz): δ=1.73 (s, 3H), 1.79 (s, 3H), 2.43 (s, 3H), 7.25-7.29 (m, 3H), 7.33-7.37 (m, 2H), 7.43-7.46 (m, 2H), 7.61-7.63 (m, 2H), 8.26 (s, 1H). ¹³C NMR (CDCl₃, 100 MHz): δ=21.1, 21.6, 21.8, 64.3, 127.4, 127.5, 127.8, 129.2, 129.4, 131.4, 138.2, 143.0, 162.4.

Compound (R_{S})-7e: [α]_{D}²⁵=−219.7° (c=1.0, CHCl₃); NMR (CDCl₃, 400 MHz): δ=1.73 (s, 3H), 1.78 (s, 3H), 6.96-7.03 (m, 1H), 7.09-7.13 (m, 1H), 7.31-7.37 (m, 1H), 7.40-7.54 (m, 9H), 8.11 (d, J=9.2 Hz, 1H). ¹³C NMR (CDCl₃, 100 MHz): δ=20.9, 21.8, 64.2, 125.2, 127.2, 127.5, 127.7, 127.8, 128.7, 130.0, 134.7, 138.1, 146.2, 163.6.

Compound (R_{S})-7f: [α]_{D}²⁵=+105.8° (c=1.0, CHCl₃); ¹H NMR (CDCl₃, 400 MHz): δ=1.70 (s, 6H), 6.49-6.50 (m, 1H), 6.83-6.84 (m, 1H), 7.22-7.30 (m, 3H), 7.36-7.38 (m, 2H), 7.57-7.58 (m, 1H), 7.99 (s, 1H). ¹³C NMR (CDCl₃, 100 MHz): δ=21.2, 21.9, 64.7, 112.3, 118.6, 127.5, 127.6, 127.8, 137.7, 146.7, 149.6, 150.6.

Compound (R_{S})-7g: [α]_{D}²⁵=−230.5° (c=1.0, CHCl₃); ¹H NMR (CDCl₃, 400 MHz): δ=0.88 (t, J=6.4 Hz, 6H), 1.73 (s, 3H), 1.74 (s, 3H), 1.81-1.91 (m, 1H), 2.16-2.21 (m, 2H), 7.31-7.33 (m, 1H), 7.35-7.41 (m, 4H), 7.72 (t, J=5.5 Hz, 1H). ¹³C NMR (CDCl₃, 100 MHz): δ=21.6, 21.8, 22.3, 22.5, 25.9, 44.6, 63.1, 127.3, 127.5, 127.8, 137.9, 169.5.

Compound (R_{S})-7h: [α]_{D}²⁵=−197.0° (c=1.0, CHCl₃); ¹H NMR (CDCl₃, 400 MHz): G=1.04-1.27 (m, 6H), 1.62-1.72 (m, 10H), 2.17-2.21 (m, 1H), 7.27-7.31 (m, 1H), 7.32-7.39 (m, 4H), 7.57 (d, J=4.6 Hz, 1H). ¹³C NMR (CDCl₃, 100 MHz): δ=21.5, 22.1, 25.2, 25.7, 28.7, 28.8, 43.8, 63.1, 127.4, 127.5, 127.7, 137.8, 172.6.

Compound (R_{S})-7i: [α]_{D}²⁵=−127.0° (c=1.0, CHCl₃); ¹H NMR (CDCl₃, 400 MHz): G=1.29 (t, J=7.3 Hz, 3H), 1.72 (d, J=3.2 Hz, 6H), 4.25-4.28 (m, 2H), 7.25-7.32 (m, 5H), 7.51 (d, 1H). ¹³C NMR (CDCl₃, 100 MHz): δ=14.0, 21.4, 22.7, 62.2, 65.9, 127.4, 127.9, 128.1, 136.6, 155.1, 160.8.

Compound (R_{S})-7j: [α]_{D}²⁵−223.6° (c=1.0, CHCl₃); ¹H NMR (CDCl₃, 400 MHz): G=1.76 (s, 3H), 1.78 (s, 3H), 2.16 (s, 3H), 7.21-7.37 (m, 5H), 7.41-7.43 (m, 3H), 7.69 (d, J=7.8 Hz, 2H). ¹³C NMR (CDCl₃, 100 MHz): δ=18.6, 19.4, 22.9, 64.5, 127.2, 127.6, 127.6, 128.1, 128.3, 131.5, 138.2, 139.2, 176.2.

TABLE 1
Results of N-(R_{S})-2-phenylpropyl-2-sulfinylimines

[chemical structure]

| | Aldehyde or | | | | |
|---|---|---|---|---|---|
| Entry | ketone | R₂ | R₃ | Product | Yield (%) |
| 1 | 6a | phenyl | H | (R_{S})-7a | 98 |
| 2 | 6b | 1-naphthyl | H | (R_{S})-7b | 97 |
| 3 | 6c | 2-Me-phenyl | H | (R_{S})-7c | 89 |
| 4 | 6d | 4-Me-phenyl | H | (R_{S})-7d | 96 |
| 5 | 6e | trans-PhCH═CH | H | (R_{S})-7e | 92 |
| 6 | 6f | 2-furyl | H | (R_{S})-7f | 92 |
| 7 | 6g | iso-butyl | H | (R_{S})-7g | 87 |
| 8 | 6h | cyclohexyl | H | (R_{S})-7h | 88 |
| 9 | 6i | —COOEt | H | (R_{S})-7i | 45 |
| 10 | 6j | phenyl | Me | (R_{S})-7j | 52 |

### Example 3

### GAP Chemistry: Efficient and Benign Asymmetric Grignard Addition onto New Chiral N-2-phenyl-2-propyl sulfinylimines

### Abstract

A new chiral (Rs)-2-phenyl-2-propyl sulfinamide has been designed and synthesized; its aldimines and ketimines have been utilized in asymmetric addition of allylmagnesium bromide, conveniently at room temperature to accomplish a series of homoallylic amines in high chemical efficiency and diastereoselectivity (>99%). The pure products are obtained by relying on benign Group Assisted Purification (GAP) method developed in our labs, which is more economical and efficient than traditional purification methods such as column chromatography and recrystallization. The absolute stereochemistry has been determined by X-ray analysis.

### Introduction

Imine chemistry has been one of the most powerful tools in modern organic synthesis to build amine moieties. Specifically, chiral amine compounds are valuable precursors in organic synthesis, as they are conveniently transformed into valuable complex natural products and pharmaceutical important drugs. Design of chiral N-protected imines (such as N-sulfinyl, N-phosphonyl, N-phosphoryl and several others) and its applications in asymmetric transformations have dramatically advanced the field of imine chemistry. Even though the imine chemistry widely studied for many years, the continuous search for chiral imines to further simplify the imine chemistry for general use is still challenging. With this goal, for the past several years we have designed several chiral N-protected imines (such as N-phosphonylimines, N-phosphinylimines, and N-phosphorylimines)) and utilized in many asymmetric transformations with good yields and diastereoselectivities without using traditional purification methods, and this process has been named Group Assisted Purification (GAP), in which the products are purified by simple washing with the common organic solvents. Some exemplary chiral N-protected imines for GAP process are shown below.

[chemical structure]

N-phosphonylimines and N-phosphinylimines shown above, were developed in our laboratories and applied for the GAP process in several asymmetric transformations such as aza-Henry, aza-Darzen, diketone addition, and others. Sulfinylimines developed by Davis, and Ellman are very useful in organic synthesis for making chiral amine compounds. We envisioned that applying the GAP concept to sulfinylimines, which can lead to i) introducing new chiral auxiliary, ii) improving the efficiency of existing transformations, and iii) simplifying the purification of the products. As a result, in the process of developing a new, efficient, and benign chiral N-protecting group for asymmetric addition reactions, we have designed the new chiral N-2-phenyl-2-propyl sulfinylimines.

Due in part to the importance homoallylic amines shown in organic synthesis, the asymmetric addition of allylmagnesium bromide was chosen as a model reaction. N-protected homoallyl amines are very useful intermediates for the wide range of pharmaceutical compounds and biologically active substances and these amines can also be transformed into nitrogen containing heterocyclic compounds such as enantiopure C₂-symmetrical trans-2,5-disubstituted pyrrolidines, which are identified to be as organocatalysts, as chiral ligands for asymmetric catalysis, and as chiral auxiliaries. Traditionally, allylation of imines through C—C bond formation by allylmetal reagents (such as allylindium, allylmagnesium and allylzinc) is used to make homoallyl amines. However, very few reactions found be successful at room temperature reaction conditions and most of the reactions require either high temperatures or very low temperature to achieve good results. We envisioned that incorporating the GAP concept to the traditional method to make homoallyl amines and developing the reaction at ambient temperatures would be more efficient in terms of time and cost. Herein, we would like to disclose our initial results towards simplifying the imine chemistry and so the synthesis of homoallylic amines with our new chiral N—(Rs)-2-phenyl-2-propylsulfinylimines.

### Results and Discussions

The synthesis of chiral (R)-2-phenyl-2-propyl sulfinamide was started from thiol 3.1. Initially, thiol 3.1 on treatment with 30% aqueous hydrogen peroxide in the presence of NaI gave the disulfide 3.2. Asymmetric oxidation of disulfide 3.2 was accomplished with 30% aqueous H₂O₂ in presence of vanadylacetoacetate and chiral ligand 3.3 yielding corresponding (R)-thiosulfinate 3.4 in good yield and enantioselectivity. The direct synthesis of sulfinamide from (R)-thiosulfinate 3.4 with Li/liq. NH₃ was failed to give desired product. After several futile attempts we examined the electrophilicity of thiosulfinate ester 3.4 by treating the same with benzylamine in presence of n-BuLi, which produced benzyl protected sulfinamide in quantitative yield. With this encouraging result, LiHMDS was reacted with thiosulfinate ester, but unfortunately bulky silylamide failed to give sulfinamide.

Then the nucleophilic substitution of freshly prepared TBDMS-NH₂ (1M solution in THF) with thiosulfinate ester in presence of n-BuLi provided TBDMS protected sulfinamide 3.5 in good yield and without racemization. The subsequent deprotection of TBDMS gave (R)-2-phenyl-2-propyl sulfinamide 3.6 in 53% overall yield and excellent enantiomeric excess (Scheme 3.1).

[chemical structure]

Next, N-2-phenyl-2-propyl-sulfinylimines were synthesized by condensation of amide with corresponding aldehyde in presence of Ti(OEt)₄. The results are shown in Table 3.1. As can be seen from Table 3.2, all types of aldehydes and ketones are viable to make their sulfinylimines. In general, good to excellent yields were achieved with aromatic and aliphatic aldehydes. In the case of acetophenone and glyoxamte aldehydes moderate yields were achieved (Table 3.1, 3.8j, 3.8 k).

TABLE 3.1
Results of N-(R_{S})-2-phenylpropyl-2-sulfinylimines

[chemical structure]

[chemical structure]

| | Aldehyde or | | | | |
|---|---|---|---|---|---|
| Entry | ketone | R₂ | R₃ | Product | Yield (%)^{a} |
| 1 | 3.7a | Phenyl | H | 3.8a | 98 |
| 2 | 3.7b | 1-Naphthyl | H | 3.8b | 97 |
| 3 | 3.7c | 2-Me-phenyl | H | 3.8c | 89 |
| 4 | 3.7d | 4-Me-phenyl | H | 3.8d | 96 |
| 5 | 3.7e | 4-F-phenyl | H | 3.8e | 98 |
| 6 | 3.7f | 2-furyl | H | 3.8f | 92 |
| 7 | 3.7g | iso-butyl | H | 3.8g | 87 |
| 8 | 3.7h | Cyclohexyl | H | 3.8h | 88 |
| 9 | 3.7i | trans-PhCH═CH | H | 3.8i | 92 |
| 10 | 3.7j | Phenyl | Me | 3.8j | 52 |
| 11 | 3.7k | —COOEt | H | 3.8k | 45^{b} |
| 12 | 3.7l | 4-NO₂-phenyl | H | 3.81 | 80^{c} |

^{a}Isolated yields after column chromatography.
^{b}4 Å M.S. were used instead of Ti(OEt)₄.
^{c}Reaction carried out at room temperature.

Initially, we began our investigation with the addition of allylmagnesium bromide to N-sulfinylimine in THF as solvent at −78° C. Though the product formed in quantitative yield, but poor diastereoselectivity (1:1) was achieved. Surprisingly, when the reaction performed at 0° C. selectivity improved to 5:1, and at room temperature the diastereoselectivity was further improved to 8:1. However, raising the reaction temperature to 50° C. did not result in any additional enhancement of diastereoselectivity. With this interesting temperature phenomenon on the reaction shown in Table 3.2, we proceeded to investigate the effect of solvents such as Toluene, Benzene, Dichloromethane, and Ether. Toluene proved to be efficient solvent in terms of good yields and high diastereoselectivities, whereas Benzene, dichloromethane gave poor yields and ether gave slightly lower diastereoselectivity.

With the optimized condition in hand, we examined the substrate scope of imines towards this addition reaction condition. The results were presented in Table 3.2, and excellent diastereoselectivities were achieved in all the cases that were studied. In general, the nature and position of the substituents of imines does not have significant effect on diastereodiscrimination. However, as expected imines tethered with reactive substituents, such as 4-nitro and —CO₂Et (Table 3.2, 3.9 k, 3.91) failed to give clean desired products. It is worthy to note that the pure homoallylic amine products were obtained in good yields without any column purification. The crude products were dried and the solid products were washed with minimum amounts of hexanes or heptanes to get pure products, and the liquid products were also obtained in more than 99% purity after washing the reaction mixture with water. Thus, the new sulfinyl auxiliary is also useful for the Group Assisted Purification process.

TABLE 3.2

| Results of the allylmagnesium Grignard addition reaction to | | | | |
|---|---|---|---|---|
| N-(R_{S})-2-phenylpropyl-2-sulfinylimines | | | | |
| [chemical structure] | | | | |
| [chemical structure] | | | | |
| [chemical structure] | | | | |
| Entry | Imine | Product | % yield^{a,b} | % de^{c} |
| 1 | 3.8a | 3.9a | Quantitative | >99 |
| 2 | 3.8b | 3.9b | Quantitative | >99 |
| 3 | 3.8c | 3.9c | 95 | >99 |
| 4 | 3.8d | 3.9d | 99 | 97 |
| 5 | 3.8e | 3.9e | 94 | 98 |
| 6 | 3.8f | 3.9f | 96 | 98 |
| 7 | 3.8g | 3.9g | Quantitative | 98 |
| 8 | 3.8h | 3.9h | 99 | 97 |
| 9 | 3.8i | 3.9i | 94 | 92 |
| 10 | 3.8j | 3.9j | 91 | >99 |
| 11 | 3.8k | 3.9k | ND | ND^{d} |
| 12 | 3.8l | 3.9l | ND | ND^{d} |

^{a}Isolated yields after washing with hexanes.
^{b}combined yields of both the diastereomers.
^{c}Diastereoselectivities calculated based on ¹H NMR of crude samples.
^{d}Clean reactions were not observed as expected, due to tethered reactive functional groups on imine.

Finally, the absolute configuration 3.9a was determined by X-ray analysis, and those of others were tentatively assigned by analogy. As shown in the figure the sulfur center determined to be R and the new chiral carbon center induced by the sulfur during addition reaction was determined as S (see X-ray structure of N-sulfinyl homoallyl amine 3.9a, below).

[chemical structure]

The deprotection of sulfinyl group of 3.9a is carried out in acidic condition to get its homoallyl amine 3.10 with ease (scheme 3.2).

[chemical structure]

The stereochemical outcome was explained with a chair like transition state, shown below, in which magnesium metal co-ordinates with both nitrogen and oxygen atoms to anchor the attack of nucleophile from Si face of imine and leads to the S stereochemical induction. The transition state for allylmagnesium bromide addition is shown below.

[chemical structure]

### Conclusions

In summary, with the goal of developing a chiral auxiliary to aid Group Assisted Purification (GAP), a new chiral N—(Rs)-2-phenyl-2-propylsulfinylimines were synthesized and utilized efficiently for the allylmagnesium Grignard addition reactions and achieved good yields and excellent diastereoselectivities. The new auxiliary enables the purification of the addition products to be easy by washing with minimum amounts of common organic solvents, which is more economical and environmental friendly. Considering the high efficiency that the present sulfinyl auxiliary exhibited, we believe the new auxiliary is useful for more green chemistry. Further studies on extension of GAP concept of sulfinylimines to other asymmetric nucleophilic addition reactions are in progress.

### Example 4

GAP Synthesis Using New and Known Chiral Sulfinylimines

[chemical structure]

[chemical structure]

In Schemes 4.1 and 4.2, R^{A}, R₂ and R₃ are as defined in the context of Formula 1001, and Nuc is any nucleophilic group. In an embodiment, a method of adding an aminoalkyl group to a nucleophilic group is provided substantially as shown in Scheme 4.1. In an embodiment, a method of adding an aminoalkyl group to a nucleophilic group is provided substantially as shown in Scheme 4.2.

GAP chemistry (Group-Assisted Purification chemistry) can be achieved by using the chiral sulfinylimines disclosed herein, as well as known chiral sulfinylimines. Therefore, when asymmetric synthesis is conducted, the use of traditional purification methods such as chromatography or recrystallization can be avoided. The pure products, often pure isomers, can be obtained simply by washing the solid crude products with organic solvents.

The characteristics of GAP chemistry require the functional groups (here, the sulfinyl group) of starting materials to enable the resulting products to have adequate solubility, i.e., the products must be dissolved in a solvent or solvents (e.g., THF, DCM, etc.) for further transformations, but should not be readily dissolved in others (e.g., hexane, petroleum ether, their mixtures with EtOAc, etc.). The functional groups should enable their attached substrates to have efficient chemical reactivity toward various species. For asymmetric reactions, the functional groups should be able to show efficient asymmetric induction and control.

All publications, patents, and patent applications cited in this specification are herein incorporated by reference as if each individual publication, patent, or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the claimed subject matter is limited solely by the scope of the following claims, including equivalents thereof.

What is claimed is:

1. A compound according to Formula 1001a or 1001b: [chemical structure]
wherein:
Y is amino or imino; and
R^{A} is lower alkyl or aryl.

2. The compound of claim 1 according to Formula Ia, Ib, IIa or IIB, or a substantially diastereomerically pure form thereof: [chemical structure]
wherein R^{A} is R₁Ph- in Formula Ia, Ib, IIa, or IIb, and each R₁ is independently hydrogen, alkyl or aryl.

3. The compound of claim 1, wherein each R₁ is independently hydrogen, methyl, isopropyl, furanyl, pyridinyl, phenyl, biphenyl, or naphthyl.

4. The compound of claim 1 according to
Formula 1002a, 1002b or a substantially diastereomerically pure form thereof: [chemical structure]
wherein:
each R₂ is independently lower alkyl, aryl, alkylaryl, arylalkenyl, alkoxycarbonyl, nitroaryl, or cycloalkyl; and
each R₃ is independently hydrogen or lower alkyl; or
Formula IIIa, IIIb or a substantially diastereomerically pure form thereof: [chemical structure]
wherein each of R₁, R₂, and R₃ is independently hydrogen, alkyl or aryl.

5. The compound of claim 4, wherein each R₁ or R₂ are independently methyl, isopropyl, furanyl, pyridinyl, phenyl, biphenyl, or naphthyl, or
wherein each R3 is independently hydrogen, methyl, isopropyl, furanyl, pyridinyl, phenyl, biphenyl, naphthyl, or cycloalkyl.

6. The compound of claim 4, wherein R₂ is phenyl, 1-napthyl, 2-methyl-phenyl, 4-methyl-phenyl, trans-Ph-CH═CH, 2-furyl, isobutyl, cyclohexyl, or —C(O)OCH₂CH₃.

7. The compound of claim 4, wherein:
R₁ is hydrogen;
R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; or
R₃ is hydrogen or methyl.

8. The compound of claim 1, in a diastereomerically pure form produced substantially as described in Scheme 1, Scheme 2, Scheme 3, Scheme 4, Scheme 3.1, Scheme 3.2, Scheme 4.1 or Scheme 4.2.

9. A method for preparing a compound of Formula Ia or Ib,
wherein the compound of Formula Ia is formed by:
a. reacting a compound of formula i2 in the presence of a compound of formula i3a to form a compound of formula i4a: [chemical structure]
b. reacting the compound of formula i4a in the presence of TBDMS-NH₂ to form a compound of formula i5a: [chemical structure]
and
c. reacting the compound of formula i5a in the presence of TBAF to form the compound of formula Ia: [chemical structure]
and
wherein the compound of Formula Ib is formed by:
a. reacting a compound of formula i2 in the presence of a compound of formula i3b to form a compound of formula i4b: [chemical structure]
b. reacting the compound of formula i4b in the presence of TBDMS-NH₂ to form a compound of formula i5b: [chemical structure]
and
c. reacting the compound of formula i5b in the presence of TBAF to form the compound of formula Ib: [chemical structure]
wherein each R₁ is independently hydrogen, alkyl or aryl.

10. The method of claim 9, wherein the reaction forming the compound of Formula Ia of step a proceeds substantially in the absence of compounds i3b and i4b,
wherein the reaction forming the compound of Formula Ia of step b proceeds substantially in the absence of compounds i4b and i5b, or
wherein the reaction forming the compound of Formula Ia of step c proceeds substantially in the absence of compounds i5b and Ib; and
wherein the reaction forming the compound of Formula Ib of step a proceeds substantially in the absence of compounds i3a and i4a,
wherein the reaction forming the compound of Formula Ib of step b proceeds substantially in the absence of compounds i4a and i5a, or
wherein the reaction forming the compound of Formula Ib of step c proceeds substantially in the absence of compounds i5a and Ia.

11. The method of claim 9, which is substantially as described in Scheme 1, Scheme 2, Scheme 3, Scheme 4, Scheme 3.1, Scheme 3.2, Scheme 4.1 or Scheme 4.2, forming a diastereomerically pure compound as described therein.

12. A method for the preparation of a compound of formula 1002a or 1002b (1002 (a, b)), formula IIIa, or IIIb,
wherein:
the compound of formula 1002 (a, b) is formed by reacting a compound of formula 1003a or 1003b (1003 (a, b)) with a compound of formula i6 to form a compound of formula 1002 (a, b): [chemical structure]
the compound of formula IIIa is formed by reacting a compound of formula Ia with a compound of formula i6 to form a compound of formula IIIa: [chemical structure]
the compound of formula IIIb is formed by reacting a compound of formula Ib with a compound of formula i6 to form a compound of formula IIIb: [chemical structure]
and
wherein each of R₁, R₂, and R₃ is independently hydrogen, alkyl or aryl.

13. The method of claim 12, wherein:
the reaction of forming compound 1002a proceeds substantially in the absence of compound 1003b,
the reaction of forming compound 1002b proceeds substantially in the absence of compound 1003a,
the reaction of forming compound IIIa proceeds substantially in the absence of compound Ib, or
the reaction of forming compound IIIb proceeds substantially in the absence of compound Ia.

14. The method of claim 12, wherein each R₁ is independently hydrogen, methyl, isopropyl, furanyl, pyridinyl, phenyl, biphenyl, or naphthyl.

15. The method of claim 12, wherein each R₂ is independently hydrogen, methyl, isopropyl, furanyl, pyridinyl, phenyl, biphenyl, or naphthyl.

16. The method of claim 12, wherein each R₃ is independently hydrogen, methyl, isopropyl, furanyl, pyridinyl, phenyl, biphenyl, naphthyl, or cycloalkyl.

17. The method of claim 12, wherein R₂ is phenyl, 1-napthyl, 2-methyl-phenyl, 4-methyl-phenyl, trans-Ph-CH═CH, 2-furyl, isobutyl, cyclohexyl, or —C(O)OCH₂CH₃.

18. The method of claim 12, wherein:
R₁ is hydrogen;
R₂ is phenyl, 1-napthyl, 2-methyl-phenyl, 4-methyl-phenyl, trans-Ph-CH═CH—, 2-furyl, isobutyl, cyclohexyl, or —C(O)OCH₂CH₃; and
R₃ is hydrogen or methyl.

19. The method of claim 12, which is substantially as described in Scheme 1, Scheme 2, Scheme 3, Scheme 4, Scheme 3.1, Scheme 3.2, Scheme 4.1 or Scheme 4.2, forming a diastereomerically pure compound as described therein.
